(19) United States
(12) Plant Patent Application Publication
(10) Pub. No.: US 2002/0178478 P1
(43) Pub. Date: Nov. 28, 2002

(54) Dahlia plant named Dapadpor

(75) Inventor: Francois Van Hee, Emkhuizen (NL)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(73) Assignee: Ball Floraplant, a division of Ball Horticultural Company

(21) Appl. No.: 09/819,868

(22) Filed: Mar. 28, 2001

(51) Int. Cl. A01H 5/00

(52) U.S. Cl. PLT/321

(57) ABSTRACT

A new and distinct Dahlia plant named ‘Dapadpor’ is provided.

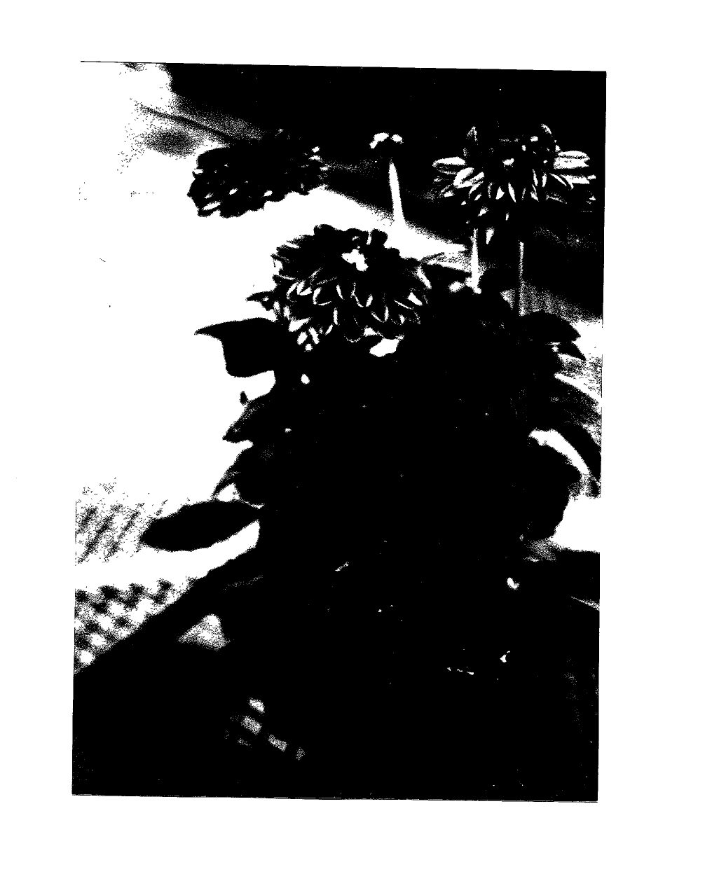

### BACKGROUND OF THE INVENTION

The present invention comprises a new and distinct Dahlia plant, botanically known as Dahlia variabilis and hereinafter referred to by the cultivar name Dapadpor.

The new Dahlia is a naturally occurring mutation or sport of the orange lowered Dapaor (U.S. Plant Pat. No. 11,701). The new Dahlia was discovered by the inventor in a controlled environment in Enkhuizen, The Netherlands, within a population of plants of Dapaor.

Asexual reproduction of the new cultivar by terminal or stem cuttings taken during 2000 at West Chicago Ill., USA has demonstrated that the characteristics of the new cultivar as herein Described are firmly fixed and are retained through successive generations of such asexual propagation.

### SUMMARY OF THE INVENTION

It was found that the cultivar of the present invention:

(a) exhibits an upright mounded habit,

(b) forms deep orange flowers and

(c) exhibits a vigorous growth habit.

Dapadpor has not been observed under all possible environmental conditions to date. Accordingly, it is possible that the phenotype may vary somewhat with variations in the environment, such as temperature, light intensity, and day length.

The new cultivar of the present invention can be compared to Dapaor and to Connie (U.S. Plant Pat. No. 6,768). However, in side by side comparisons, Dapadpor exhibits darker orange ray florets, and slightly smaller inflorescences. When compared to Connie, Dapadpor exhibits smaller flowers, and different stem coloration and pubescence as detailed in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown for nine weeks in a greenhouse at West Chicago, Ill., USA.

### DETAILED BOTANICAL DESCRIPTION

The chart used in the identification of colors described herein is the R.H.S Colour Chart of The Royal Horticultural Society, London, England. The color values were determined on Mar. 6, 2001 in West Chicago, Ill. The readings were taken between 10:00 and 11:45 p.m. under natural light conditions. The plants were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night. Plants were pinched at three weeks after planting. The following measurements and descriptions were taken at 8 weeks after planting.

Classification:

Botanical.—Dahlia variabilis cultivar Dapadpor.

Commercial.—Dahlia.

Parentage: Sport or mutation of Dapaor

Propagation:

Type cutting.—Terminal tip.

Time to Initiate Roots.—Approximately 7 to 10 days.

Time to develop roots.—Approximately 14 to 21 days.

Root description.—Fibrous and well branched.

Tubers.—Will form under short day conditions of at least 13 to 14 hours of darkness.

Plant description:

General Appearance and Form.—Upright and mounded, vigorous.

Height above pot.—Approximately 22 cm.

Width.—Approximately 18 cm.

Branching habit.—Freely branching. Pinching helps to initiate branching.

Branch description.—Diameter: Approximately 6 mm. Color: Slightly darker than 144A. Texture: Smooth. Internode length: Approximately 2 cm.

Foliage description:

Form.—Mature foliage simple, occasionally trifoliate.

Shape.—Ovate.

Margin.—Serrate.

Apex.—Acuminate.

Base.—Attenuate.

Arrangement.—Opposite.

Surface.—Upper surface slightly rugose.

Color of mature foliage.—upper surface: Closest to 139A with veins of 146C.

Color of mature foliage.—lower surface: Closest to 191A with veins of 146B.

Size.—Single mature leaves are approximately 7.1 cm in length, approximately 8 cm in width. Mature trifoliate leaves are approximately 7.6 cm in length and approximately 8 cm in width. Petiole is 5.5 cm in length, 3 mm in diameter and 146B in color

Flower description:

Outdoor Flowering Habit.—Dapadpor is freely flowering under outdoor growing conditions with substantially continuous blooming from spring through fall.

Appearance.—Fully double arising from leaf axils on strong peduncles. Disc and ray florets arranged acropetally on a capitulum.

Flower bud.—Shape: Round. Length: Approximately 1.25 cm. Diameter: Approximately 1.25 cm. Color: 144A.

Inflorescence size.—Approximately 5.3 cm. Disc diameter: Approximately 1 cm.

Ray florets.—Quantity: Approximately 45. Shape: Elongated, cupped. Length: Approximately 2.2 cm. Apex: Pointed to slightly rounded. Base: Attenuate. Margin: Entire. Texture: Smooth. Color: Fully opened, upper surface: 34A. Fully opened, lower surface between 167A and 167B.

Disc florets.—Shape: Elongated, cylindrical. Color: 14B.

Peduncle.—Strong, erect. Length: Approximately 3.4 cm.

Involucral bracts.—Quantity: 5. Shape: Diamond-shaped. Tip: Broadly acute. Margin: Entire. Texture: Smooth. Color: 139A.

Reproductive Organs.—Androecium—On disc florets. Anthers approximately 3 mm in length.

Gynoecium.—On disc and ray florets. Pistil length: Approximately 1 cm. Pollen color is 14A.

Seed production: Seed production has not been observed.

Disease resistance: Disease resistance has not been observed and is not claimed.

TABLE A

| CHARACTER- | | | |
|---|---|---|---|
| ISTIC | DAPADPOR | DAPAOR | CONNIE |
| FLOWER COLOR | 34AFADING | 33A FADING | 43A |
| | TO 32B | TO 168C | |
| | AT EDGES | AT APEX | |
| INFLORESCENCE | 5.3 | 8 | 8 |
| DIAMETER-CM | | | |
| STEM COLOR | DARKER | 144A | 144A WITH |
| | THAN 144A | | SLIGHT |
| | | | ANTHOCYANIN |
| | | | COLORATION, |
| | | | ESPECIALLY |
| | | | AT NODES |
| STEM | NONE | NONE | SLIGHT |
| PUBESCENCE | | | PUBESCENCE |
| | | | AT NODES |

I claim:

1. A new and distinct cultivar of Dahlia named Dapadpor substantially as herein shown and described, which:
(a) exhibits an upright mounded habit,
(b) forms deep orange flowers and
(c) exhibits a vigorous growth habit.
